(19) United States
(12) Patent Application Publication
(10) Pub. No.: US 2021/0315919 A1
(43) Pub. Date: Oct. 14, 2021

(54) SYSTEM FOR IMPROVING RECOVERY FROM SURGERY

(71) Applicant: The Trustees of Indiana University, Indianapolis, IN (US)

(72) Inventors: William Arthur Wooden, Clayton, IN (US); Nancy M. Strnge, Indianapolis, IN (US)

(21) Appl. No.: 17/229,000

(22) Filed: Apr. 13, 2021

Related U.S. Application Data
(60) Provisional application No. 63/009,186, filed on Apr. 13, 2020.

(51) Int. Cl. A61K 31/716; A61K 31/198; A61K 9/00; A61K 45/06

(52) U.S. Cl. CPC A61K 31/716; A61K 45/06; A61K 9/0053; A61K 31/198

(57) ABSTRACT

Methods for pre-surgical medical prehabilitation of a patient prior to a date of surgery to improve post-surgical recovery include directing oral administration of an immunonutritional supplement to the patient during a first time period prior to the surgery date; directing administration of pulmonary preconditioning treatments to the patient during a second time period prior to the surgery date; directing administration of a bacterial decontamination protocol to the patient during a third time period prior to the surgery date; and directing oral administration of a carbohydrate loading and hydration protocol to the patient during a fourth time period prior to the surgery date. Kits for completing a pre-surgical medical prehabilitation protocol prior to a date of surgery to improve post-surgical recovery include at least five doses of an immunonutritional supplement; a spirometer and/or a set of instructions for performing pulmonary preconditioning treatments; an anti-bacterial body wash composition; an anti-bacterial nasal cavity wash composition; and at least three doses of a carbohydrate loading and hydration composition.

### CROSS REFERENCE TO RELATED APPLICATIONS

This application claims priority to U.S. Provisional Patent Application No. 63/009,186 filed on Apr. 13, 2020, which is incorporated by reference herein in its entirety.

### BACKGROUND

The general field of the present disclosure is provider-directed (e.g., physician-directed) engagement in medical prehabilitation.

Healthcare systems in the United States and around the world have been challenged to improve quality of patient care while reducing harm and cost. Historically, medical care has been delivered reactively, treating patients in reaction to an injury, illness, disease state or other acute need for medical care. Programs have been designed to attempt to improve care after a patient is hospitalized, missing opportunities to be proactive and engage the patient and his or her providers in a pre-habilitative manner. In the current state of medical care, providers and patients are too often placed in position of “catch up,” which results in higher cost and higher risk of adverse outcomes.

With regard to recovery from surgeries, positive outcomes and optimal recoveries are hindered when surgery patients enter into surgeries in suboptimal physical condition. Efforts to address this problem have resulted in the development of enhanced recovery programs, examples of which have been introduced in the United States and in Europe; however, these programs are quite cumbersome, costly, and difficult to implement due to their complexity. They also fail to address some of the root causes of the surgery patients' suboptimal physical condition, including malnutrition and poor immunonutrition.

There remains a need for solutions to the above problems, including a need for systems and methods to reduce harm and improve positive outcomes and recoveries from surgery that are easy to implement for most patients, that improve the trajectory of the patient's health and improve the health care system overall. The present disclosure addresses these needs.

### SUMMARY

The present disclosure provides systems, methods, processes and kits for improving surgical outcomes and recoveries and reducing harm.

In one aspect of the disclosure, there is provided a method for pre-surgical medical prehabilitation of a patient prior to a surgery to improve post-surgical recovery that includes: (a) directing oral administration of at least one dose of an immunonutritional supplement per day to the patient during a first time period prior to the surgery date; (b) directing administration of at least two pulmonary preconditioning treatments per day to the patient during a second time period prior to the surgery date; (c) directing administration of a bacterial decontamination protocol to the patient during a third time period prior to the surgery date; and (d) directing oral administration of a carbohydrate loading and hydration protocol to the patient during a fourth time period prior to the surgery date. In some embodiments, the first time period is from about three to about seven days. In some embodiments, the second time period is from about 1 to about 30 days. In some embodiments, the third time period is from about one to about three days. In some embodiments, the fourth time period is from about one to about two days.

In some embodiments, the method further includes one or more of conducting a physical assessment of the patient from 1 to about 30 days prior to the surgery date, prescribing to the patient a pre-surgical exercise protocol during a fifth time period prior to the surgery date and providing to the patient educational information regarding smoking cessation and/or assistance for smoking cessation. In some embodiments, the method further includes directing oral administration of at least one dose of an immunonutritional supplement per day to the patient during a post-surgery time period; directing administration of at least two pulmonary preconditioning treatments per day to the patient during the post-surgery time period; and directing oral administration of a carbohydrate loading and hydration protocol to the patient during the post-surgery time period.

In another aspect, the disclosure provides a kit for completing a pre-surgical medical prehabilitation protocol prior to a date of surgery to improve post-surgical recovery that includes (a) at least five doses of an immunonutritional supplement; (b) a member selected from the group consisting of a spirometer, a set of instructions for performing pulmonary preconditioning treatments and a combination thereof; (c) an anti-bacterial body wash composition; (d) an anti-bacterial nasal cavity wash composition; and (e) at least three doses of a carbohydrate loading and hydration composition. In some embodiments, the kit further includes one or more of an oral cavity wash composition, a checklist including schedule information for each prescribed action in the pre-surgical medical prehabilitation protocol and a structure for tracking completion of each prescribed action, a nutritional supplement customized to a patient following a nutritional evaluation of the patient, educational information, and availability of a referral for smoking cessation.

In some embodiments, the kit includes a software application loaded on a computer or a hand-held electronic device, the application executable to generate a prompt for each action to be taken by a patient in accordance with a pre-surgical medical prehabilitation protocol. In some embodiments, the application is executable to notify a user that a time period for the patient to take such action has opened. In some embodiments, the application is executable to record an input by the user indicating that the action has been completed by the patient. In some embodiments, the application is further executable to communicate user inputs to a health care provider to facilitate tracking of the patient's compliance with the pre-surgical medical prehabilitation protocol.

In a further aspect of the disclosure, there is provided a method for optimizing the health of a person that includes: (a) directing oral administration of at least one dose of an immunonutritional supplement per day to the person during a health optimizing period; (b) directing administration of at least two pulmonary preconditioning treatments per day to the person during the health optimizing period; and (c) directing oral administration of a carbohydrate loading and hydration protocol to the person during the health optimizing period.

Further features, characteristics and embodiments of the present disclosure will be apparent from the detailed description herein.

### DETAILED DESCRIPTION

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

The present disclosure relates to systems, methods, processes and kits, which are referred to herein individually and collectively as POWERR programs (Pre-Operative Wellness and Enhanced Rapid Recovery), that provide documented benefits to the surgical patient population. The POWERR programs incorporate multiple provider-directed preparatory actions as sequential pre-surgery steps to improve nutrition, immunonutrition, pulmonary function and reduce infection, and to engage the patient in improving his or her baseline health regardless of health status at the time of program initiation. The programs are particularly beneficial to patients requiring elective surgery; however, the programs also are beneficial to all other surgery patients and also to patients dealing with illness, hospitalizations, and other chronic conditions. Even patients who are quite fit can improve outcomes, and improve overall health, by following the stepwise process outlined in in the POWERR programs. In addition to improving surgery outcomes, the methods, processes, kits and systems disclosed herein have the potential of improving the efficacy of cancer therapies, as well as other therapies such as cardiac rehabilitation, myocardial recovery, neurological and musculoskeletal recovery including fracture in muscle healing, and regeneration.

Preventive medicine offers substantial opportunities to improve quality and reduce complications within healthcare. The work leading to the present disclosure identified that 75% or more of the patients presenting to a large healthcare system had three or more risk factors for complications. Prior to the program disclosed herein, there were no effective programs in place to define or implement steps to mitigate such risks prior to the patient's arrival to the hospital. POWERR programs disclosed herein achieve this result by implementing pre-surgical steps that have post-surgery benefits, including, for example, reducing post-operative infections, reducing hospital length of stay and reducing the need for complex wound care support. The POWERR programs described herein include elements to address key risk factors for complications that arise during hospitalization and surgery, such as, for example, surgical site infections, venous clot formation, deep wound infections, catheter/bladder/urinary tract infections, and colitis. Following the program protocols also improves wound healing and other elements of recovery, thereby reducing a patient's length of stay and possible readmission following hospitalization.

POWERR is designed to support the patient, care givers and health care providers by providing a sequence of specific evidence-based steps by which a patient engages in individual provider-directed activities designed to improve his or her health, reduce his or her risk profile for surgery and improve his or her recovery. This program is not limited to reducing the risk of surgery but is equally adaptable to help patients recover after surgery, injury or a lifestyle disease. The elements within the program are useful and adaptable for patients with significant medical issues and those patients who have a better health risk profile.

In one aspect, this disclosure provides a method for pre-surgical medical prehabilitation of a patient prior to a date of surgery to improve post-surgical recovery that includes: (a) directing oral administration of at least one dose of an immunonutritional supplement per day to the patient during a first time period; (b) directing administration of at least two pulmonary preconditioning treatments to the patient per day during a second time period; (c) directing administration of a bacterial decontamination protocol to the patient during a third time period prior to the surgery date; and (d) directing oral administration of a carbohydrate loading and hydration protocol to the patient during a fourth time period prior to the surgery date. In some embodiments, the first time period is from about three to about seven days. In some embodiments, the second time period is from about 1 to about 30 days. In other embodiments, the second time period is from about 1 to about 14 days. In some embodiments, the third time period is from about one to about three days. In some embodiments, the fourth time period is from about one to about two days. In preferred embodiments, each of the first, second, third and fourth time period is a time period immediately prior to the surgery date.

With regard to directing oral administration of at least one dose of an immunonutritional supplement per day to the patient, it is known that many forms of stress on a person, including illness, trauma and surgery, result in major suppression of the immune system. Reversal of the immunosuppression can be accomplished using key nutritional and/or pharmacologic therapies involving arginine; protein synthesis; synthesis of nucleotides, DNA and RNA; and modulating adverse inflammatory processes. In some embodiments, the method includes directing oral administration of at least two doses of an immunonutritional supplement per day to the patient during the first time period. In other embodiments, the method includes directing oral administration of at least three doses of an immunonutritional supplement per day to the patient during the first time period.

In some embodiments, the immunonutritional supplement is an arginine supplement. In some embodiments, a dose of an immunonutritional supplement comprises at least about 4 to about 15 mg of arginine. The arginine can be provided, for example, as L-arginine hydrochloride (also referred to as L-arginine), which is commercially available.

In other embodiments, the immunonutritional supplement includes one or more of arginine, other amino acids, at least one Omega-3 fatty acid, and one or more nucleotides. Key elements to immune recovery include improvement of T cell function; reduction of immune suppression from Myeloid derived suppressor cells (MDSC's); increase of arginine availability; augmentation of nitric oxide production, which can be enhanced via arginine; supplementation of nucleotides in direct or indirect biologically available forms, such as from protein metabolic substrate (pyrimidine purine/DNA-RNA building blocks); and modulation of adverse inflammatory processes (e.g., adverse inflammatory cytokines and prostaglandins) with Omega-3 fatty acids.

Each of arginine, other amino acids, at least one Omega-3 fatty acid, one or more nitrates and one or more nucleotides, can contribute to positive effects and each of these substances can be provided to a patient individually in some embodiments. In other embodiments, however, a combination of these immunonutritional supplements is provided. Administration of an immunonutritional supplement in accordance with the disclosed methods demonstrates superior and measurable patient benefit by reduction in surgical site infections, reduction in hospital length of stay and improved time to wound healing by increasing the bioavailability of Arginine to support T cell function and recovery, increased nitric oxide production, support cellular synthesis and recovery and modulating the inflammatory response. One combination immunonutritional supplement that is suitably used in the disclosed methods and that is commercially available is Nestle IMPACT AR®, which includes arginine, Omega-3 fatty acids from fish oil and nucleotides. In some embodiments in which Nestle IMPACT AR® is administered, a dose of the immunonutritional supplement comprises from about 4 to about 8 fluid ounces of Nestle IMPACT AR® or about 6 fluid ounces of Nestle IMPACT AR®.

With regard to directing administration of at least two pulmonary preconditioning treatments per day to the patient during the second time period, in a preferred embodiment one pulmonary preconditioning treatment is in the morning and one pulmonary preconditioning treatment is in the evening on each respective date.

In some embodiments, each of the pulmonary preconditioning treatments comprises a treatment selected from a deep breathing session and a spirometer treatment session. In some embodiments, a deep breathing session includes a series of successive deep breaths such as, for example, a series of five or more deep breaths. As used herein, the term “deep breath” refers to an inhalation and exhalation in which the patient inhales to a degree sufficient to effect a progressive improvement in/of his or her lung capacity. In some embodiments, a deep breathing session includes a series of from about seven to about thirteen deep breaths followed by from one to five hard coughs. In one embodiment, a deep breathing session includes a series of ten deep breaths followed by three hard coughs. In other embodiments, a pulmonary preconditioning treatment includes a session using a spirometer or other lung function improvement device. In some embodiments, the number and/or the duration of pulmonary preconditioning treatments is increased each day of the second time period.

With regard to directing administration of a bacterial decontamination protocol to the patient during the third time period, in some embodiments, the bacterial decontamination protocol comprises a nasal antibacterial treatment from about two to about four times per day during the third time period. In some embodiments, the nasal antibacterial treatment includes spraying or swabbing an antibacterial composition into contact with the patient's nasal cavity surfaces to eliminate or reduce the number of microbes on such surfaces. The frequency and timing may differ based on the product and therapeutic recommendations provided by the FDA and/or a healthcare provider. Antibacterial compositions suitable for such use include, for example, Mupirocin and Provadine-iodine, each of which is commercially available.

In some embodiments, the bacterial decontamination protocol comprises an antibacterial body wash at least once per day during the fourth treatment period. In some embodiments, the antibacterial body wash includes washing the patient's body (not face) with a hospital grade soap to eliminate or reduce the number of microbes on the patient's skin surfaces. Antibacterial compositions suitable for such use include, for example, Hibiclens®, chlorhexidine gluconate, and iodophors, each of which is commercially available.

In certain embodiments, the bacterial decontamination protocol is performed at least the day before the surgery date and the morning of surgery. In other embodiments, the bacterial decontamination protocol is performed at least two consecutive days before the surgery date and the morning of surgery.

With regard to directing oral administration of a carbohydrate loading and hydration protocol to the patient during the fourth time period, in some embodiments, the carbohydrate loading and hydration protocol comprises at least three doses of carbohydrate supplements per day during the fourth time period. In other embodiments, the carbohydrate loading and hydration protocol comprises at least three doses of a carbohydrate-containing product that includes maltodextrin or one or more other source of semi-complex carbohydrates. For diabetic patients, a hydration-only product can be provided to limit delivery of excess carbohydrates. In some embodiments, the carbohydrate loading and hydration protocol comprises about 15 to about 25 ounces of G Series Gatorade® the afternoon before the surgery date, about 15 to about 25 ounces of G Series Gatorade® the evening before the surgery date, and about 7 to about 17 ounces of G Series Gatorade® the morning of the surgery date (at least three hours before surgery).

In other embodiments of the pre-surgical medical prehabilitation method, the method further includes conducting a physical assessment of the patient from 1 to about 30 days prior to the surgery date. In such embodiments, the physical assessment provides an opportunity to engage the patient in improving his or her baseline health regardless of health status at the time of program initiation. Experience has shown that close engagement with and evaluation of the patient by health care providers in a collaborative process, beginning with such a physical assessment, provides the best opportunity to prepare the patient for surgery and recovery and has the potential of helping establish a healthier future for that patient and his or her family. Thus, in some embodiments, the method includes a collaborative engagement with and preoperative physical assessment of the patient by one or more members of his or her health care provider team. This embodiment also enables health care providers to identify patients who have a difficult time coping with a disease, illness or upcoming surgery and can benefit from step-by-step instructions on how to manage their health improvement process, becoming a patient who takes ownership of his or her health care process. In other embodiments, the method includes an education process with the patient, which encourages the patient to engage with health improvement before and after a surgery.

In some embodiments, the patient's physician and care team work collaboratively to provide a thorough physical assessment of the patient. In some embodiments physical assessment includes one or more of, and preferably all of, validation of the patient's medical history, determination of the medications that the patient is taking, determining the patient's nutritional status and determining the patient's functional capacity. Preferably, the physical assessment is completed by collaboration between the patient's primary care provider, specialty care provider, surgeon, anesthesiologist and hospitalist, which allows for the collection and organization of the patient's health status to create an optimized pre-surgical medical prehabilitation protocol for the patient.

In this embodiment, once the patient has completed an initial physical assessment, the information obtained through the physical assessment, together with the timeline to surgical intervention, informs the establishment of the time lines and schedules within the pre-surgical medical prehabilitation protocol. A POWERR protocol for a patient can be designed for completion in a short period of time, such as 5 to 6 days; however, it is desirable to complete the physical assessment in this embodiment up to a month or more before the surgery date so that any special circumstances learned through the physical assessment can be addressed and so that optional additional elements, such as an exercise protocol, smoking cessation and the like can be implemented as a part of the patient's pre-surgical medical prehabilitation method.

In other embodiments of the pre-surgical medical prehabilitation method, the method further includes prescribing to the patient a pre-surgical exercise protocol during a fifth time period prior to the surgery date. Preferably, the fifth time period is a time period of at least one week, more preferably at least two weeks, still more preferably at least three weeks and even more preferably at least four weeks. The purpose of a pre-surgical exercise protocol is to increase strength and overall physical fitness prior to the date of surgery. In some embodiments, the exercise protocol comprises walking about 20 to about 40 minutes a day for at least 5 days per week. In some embodiments, the exercise protocol comprises performing a cardiovascular workout about 15 to about 25 minutes at least 3 times per week. In still other embodiments, the exercise protocol comprises both walking about 20 to about 40 minutes a day for at least 5 days per week and performing a cardiovascular workout about 15 to about 25 minutes at least 3 times per week. In some embodiments, the cardiovascular workout is effective to elevate the patient's heart rate by at least 30 beats per minute for at least 15 minutes. The cardiovascular workout can include, for example, jogging, running, an aerobic workout, strength training and combinations thereof. In other embodiments, in which the surgery is of a certain type that would be benefitted by strengthening of specific muscles and/or muscle groups, the exercise protocol may include exercises aimed at strengthening those respective muscles and/or muscle groups.

In other embodiments of the pre-surgical medical prehabilitation method, the method further includes providing to the patient at least one of educational information and assistance for smoking cessation.

In some embodiments, the pre-surgical physical assessment includes a nutritional evaluation. In some embodiments, the nutritional evaluation includes blood tests. In other embodiments, the nutritional evaluation includes querying the patient concerning parameters known to indicate nutrition risk factors, such as, for example, involuntary weight loss, decreased oral intake and Body Mass Index. Identifying malnourished patients prior to elective surgeries allows for nutrition interventions that are documented to improve adverse outcomes. When a pre-surgical assessment identifies that the patient has one or more nutrition risk factor, the patient is identified as a high-risk patient and the method can be expanded to include a nutrition intervention. For example, in a preferred embodiment, the method includes a full nutrition assessment and development of a nutrition care plan (NCP) to optimize patient outcome. This preferably includes assessment of all variables that preclude adequate nutrient intake, including medical circumstances, medications, social factors, financial factors and physical issues. Because frequent occurrence of micronutrient deficiencies (5-50% prevalence) can result in adverse events during a surgical treatment process, a full nutrition assessment and implementation of an NCP for these issues is included in certain preferred methods.

If the physical assessment reveals that the patient has lost weight in an amount of more than 10 pounds without concerted effort, the pre-surgical medical prehabilitation method preferably further includes educating the patient regarding healthy improvements in his or her oral, enteral or parenteral intake of nutrition. In some embodiments, if such patient is not diabetic, the pre-surgical medical prehabilitation method further includes administering to the patient at least three servings per day of a nutrition drink, such as, for example, Ensure® or Boost Plus®. In some embodiments, if such patient is diabetic, the pre-surgical medical prehabilitation method further includes administering to the patient at least three servings per day of a nutrition drink that is suitable for a diabetic person, such as, for example, Boost Glucose Control® or Glucerna®. In either case, the patient can cease taking the nutrition drink when he or she begins taking the immunonutritional supplement according to the pre-surgical medical prehabilitation (typically five days prior to the surgery date).

Patients with higher risk profiles based on medical history and physical assessment will benefit from longer programs than patients who present with a lower risk profile. The pre-surgical medical prehabilitation methods described herein are suitable and adaptable to all patient populations; however, a few special circumstances should be kept in mind. Certain patient populations, such as, for example, patients with glactasemia, post-transplant patients, pregnant patients, pediatric patients under the age of 13, patients with known contraindications to any of the elements or products within the program, should be excluded from the program unless a consulting physician expressly authorizes its use. In other instances, certain modifications to the method may be appropriate for such patients, and possibly patients in other at-risk categories.

It should be appreciated that the various processes described herein may allow for the management of patient engagement with the program elements/process and communication of that progress and participation/engagement to the provider. Traditional forms of communication such as face-to-face patient education using written print and other mediums have proven to be reasonably effective but also documented to show poor retention by the patient, caregivers, family and others. To advance patient engagement and thus effectiveness, the program in some embodiments includes a dynamic, mobile software application that can be used on smartphones and other mobile computing devices. Further, in some embodiments, landline and/or paper backups may be included in parallel processes. It should be appreciated that the software application loaded on a computer or a hand-held electronic device, can provide for standard as well as patient-specific adaptations to optimize a patient's engagement with the program as well as optimizing his or her medical condition and reporting back to the providers during the process at various stages (e.g., pre-interventional, immediately following intervention, end of a long-term follow-up, etc.). This can be done in single/unidirectional communication to the patient or bidirectional communication with those parties key to the outcome, who can offer support for the patient through indirect/direct feedback, optionally augmented with video engagement, including providing answers to process and patient-specific questions, monitoring progress and standalone monitoring as needed.

It should be appreciated that, in preferred embodiments, the software application and associated system is HIPPA secure and cloud/server managed. In some embodiments, the application further supports download and capture into the EMR for documentation, quality and data management as well as supporting liability monitoring/documentation and proactive care modification. In some embodiments, a software application loaded on a computer or a hand-held electronic device, either a standard customize format, is executable to generate or provide a prompt for each action to be taken by a patient in accordance with a pre-surgical medical prehabilitation protocol. In some embodiments, the application is executable to notify a user that a time period for the patient to take such action has opened. In some embodiments, the application is executable to record an input by the user indicating that the action has been completed by the patient.

In some embodiments, the software application is further executable to communicate user inputs to a health care provider to facilitate tracking of the patient's compliance with the pre-surgical medical prehabilitation protocol. In alternate embodiments, the application is executable to communicate an alert to a health care provider upon occurrence of predetermined and preset default parameters, such as, for example, when the patient fails to record one or a certain other number of inputs showing that actions under the protocol have been completed. In addition to enabling the patient's health care providers to collect data regarding compliance with the protocol, the application of such embodiments also fosters compliance and increases positive outcomes by enabling the health care providers to follow up with the patient in the event of such an alert that suggests lack of compliance with the protocol by the patient.

In some embodiments, the same software application or a different software application can be engaged with a platform to query a patient concerning parameters known to indicate nutrition risk factors, such as, for example, involuntary weight loss, decreased oral intake and Body Mass Index. Alternatively or in addition, the same software application or a different software application can be engaged with a platform to query a patient concerning other parameters, including but not limited to, patient satisfaction and wellbeing, medical and social data relative to their care, delivery of health care, commercial and/or research questions, and/or other information/data. This can be directed to a focused specific point in time for a patient such as in preparation for surgery but also expanded to manage more global needs before, during and/or after an event of care, which provides a foundation for improved health and health care engagement with providers and one's self. Examples might include nutrition risk factors, such as, for example, involuntary weight loss, decreased oral intake, and Body Mass Index. Patient directed data collection and collaborative questioning may occur in person, via pre or post arrival, paper or electronic communication to capture and allow for assessment and management. This also identifies patients prior to surgery who have nutritional deficits and enables a provider to prescribe early nutrition interventions.

Although the application is described herein primarily as being a smartphone application, it should be appreciated that the application may be embodied as any type of application suitable for performing the functions described herein. In particular, in some embodiments, the application may be embodied as a mobile application (e.g., a smartphone application), a cloud-based application, a web application, a thin-client application, and/or another type of application. For example, in some embodiments, an application may serve as a client-side interface (e.g., via a web browser) for a web-based application or service.

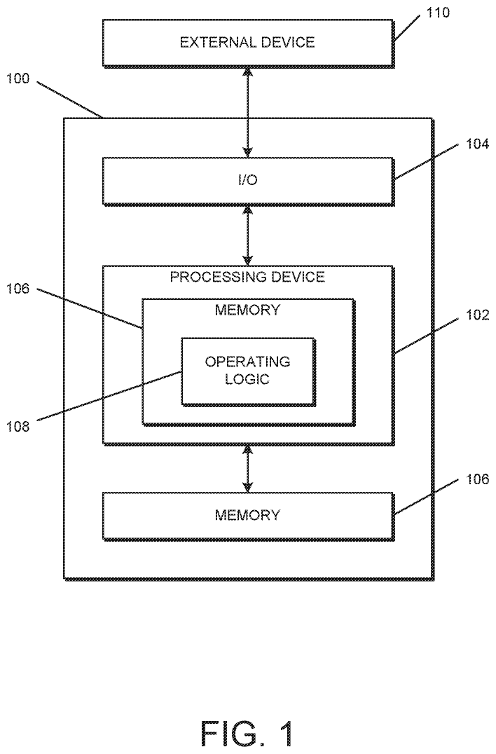

It should be further appreciated that the computing device executing the application may be embodied as any type of computing device capable of doing so. For example, in some embodiments, the computing device may be similar to the computing device 100 of FIG. 1. Referring now to FIG. 1, a simplified block diagram of at least one embodiment of a computing device 100 is shown. Depending on the particular embodiment, the computing device 100 may be embodied as a mobile computing device, cellular phone, smartphone, wearable computing device, personal digital assistant, desktop computer, laptop computer, tablet computer, notebook, netbook, Ultrabook™, Internet of Things (IoT) device, server, processing system, and/or any other computing, processing, and/or communication device capable of performing the functions described herein.

The computing device 100 includes a processing device 102 that executes algorithms and/or processes data in accordance with operating logic 108, an input/output device 104 that enables communication between the computing device 100 and one or more external devices 210, and memory 106 which stores, for example, data received from the external device 210 via the input/output device 104.

The input/output device 104 allows the computing device 100 to communicate with the external device 110. For example, the input/output device 104 may include a transceiver, a network adapter, a network card, an interface, one or more communication ports (e.g., a USB port, serial port, parallel port, an analog port, a digital port, VGA, DVI, HDMI, FireWire, CAT 5, or any other type of communication port or interface), and/or other communication circuitry. Communication circuitry of the computing device 100 may be configured to use any one or more communication technologies (e.g., wireless or wired communications) and associated protocols (e.g., Ethernet, Bluetooth®, WiMAX, etc.) to bring about such communication depending on the particular computing device 100. The input/output device 104 may include hardware, software, and/or firmware suitable for performing the techniques described herein.

The external device 110 may be any type of device that allows data to be inputted or outputted from the computing device 100. For example, in various embodiments, the external device 110 may be embodied as a server (e.g., a cloud-based server). Further, in some embodiments, the external device 110 may be embodied as another computing device, switch, diagnostic tool, controller, printer, display, alarm, peripheral device (e.g., keyboard, mouse, touch screen display, etc.), and/or any other computing, processing, and/or communication device capable of performing the functions described herein. Furthermore, in some embodiments, it should be appreciated that the external device 110 may be integrated into the computing device 100.

The processing device 102 may be embodied as any type of processor(s) capable of performing the functions described herein. In particular, the processing device 102 may be embodied as one or more single or multi-core processors, microcontrollers, or other processor or processing/controlling circuits. For example, in some embodiments, the processing device 102 may include or be embodied as an arithmetic logic unit (ALU), central processing unit (CPU), digital signal processor (DSP), and/or another suitable processor(s). The processing device 102 may be a programmable type, a dedicated hardwired state machine, or a combination thereof. Processing devices 102 with multiple processing units may utilize distributed, pipelined, and/or parallel processing in various embodiments. Further, the processing device 102 may be dedicated to performance of just the operations described herein, or may be utilized in one or more additional applications. In the illustrative embodiment, the processing device 102 is programmable and executes algorithms and/or processes data in accordance with operating logic 108 as defined by programming instructions (such as software or firmware) stored in memory 106. Additionally or alternatively, the operating logic 108 for processing device 102 may be at least partially defined by hardwired logic or other hardware. Further, the processing device 102 may include one or more components of any type suitable to process the signals received from input/output device 104 or from other components or devices and to provide desired output signals. Such components may include digital circuitry, analog circuitry, or a combination thereof

The memory 106 may be of one or more types of non-transitory computer-readable media, such as a solid-state memory, electromagnetic memory, optical memory, or a combination thereof. Furthermore, the memory 106 may be volatile and/or nonvolatile and, in some embodiments, some or all of the memory 106 may be of a portable type, such as a disk, tape, memory stick, cartridge, and/or other suitable portable memory. In operation, the memory 106 may store various data and software used during operation of the computing device 100 such as operating systems, applications, programs, libraries, and drivers. It should be appreciated that the memory 106 may store data that is manipulated by the operating logic 108 of processing device 102, such as, for example, data representative of signals received from and/or sent to the input/output device 104 in addition to or in lieu of storing programming instructions defining operating logic 108. As shown in FIG. 1, the memory 106 may be included with the processing device 102 and/or coupled to the processing device 102 depending on the particular embodiment. For example, in some embodiments, the processing device 102, the memory 106, and/or other components of the computing device 100 may form a portion of a system-on-a-chip (SoC) and be incorporated on a single integrated circuit chip.

In some embodiments, various components of the computing device 100 (e.g., the processing device 102 and the memory 106) may be communicatively coupled via an input/output subsystem, which may be embodied as circuitry and/or components to facilitate input/output operations with the processing device 102, the memory 106, and other components of the computing device 100. For example, the input/output subsystem may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations.

The computing device 100 may include other or additional components, such as those commonly found in a typical computing device (e.g., various input/output devices and/or other components), in other embodiments. It should be further appreciated that one or more of the components of the computing device 100 described herein may be distributed across multiple computing devices. In other words, the techniques described herein may be employed by a computing system that includes one or more computing devices. Additionally, although only a single processing device 102, I/O device 104, and memory 106 are illustratively shown in FIG. 1, it should be appreciated that a particular computing device 100 may include multiple processing devices 102, I/O devices 104, and/or memories 106 in other embodiments. Further, in some embodiments, more than one external device 110 may be in communication with the computing device 100.

In another aspect of the disclosure, there is provided a kit for completing a pre-surgical medical prehabilitation protocol prior to a date of surgery to improve post-surgical recovery. In some embodiments, the kit includes (a) at least five doses of an immunonutritional supplement; (b) a pulmonary preconditioning aid; (c) an anti-bacterial body wash composition; (d) an anti-bacterial nasal cavity wash composition; and (e) at least three doses of a carbohydrate loading and hydration composition.

In some embodiments of the kit, the pulmonary preconditioning aid comprises a spirometer. In other embodiments, the pulmonary preconditioning aid comprises a set of instructions for performing pulmonary preconditioning treatments. In some embodiments, the set of instructions for performing pulmonary preconditioning treatments comprises instructions for performing deep breathing sessions. In some embodiments of the kit, the immunonutritional supplement comprises an arginine supplement. In some embodiments of the kit, the immunonutritional supplement comprises an arginine-based immunonutritional supplement. In some embodiments of the kit, the carbohydrate loading and hydration composition comprises a carbohydrate-containing product that includes maltodextrin or one or more other source of semi-complex carbohydrates. For diabetic patients, a hydration-only product can be provided to limit delivery of excess carbohydrates. . In some embodiments of the kit, the kit further comprises a nutritional supplement customized to a patient following a nutritional evaluation of the patient. In some embodiments of the kit, the kit further comprises at least one of educational information for smoking cessation and a smoking cessation product.

In some embodiments of the kit, the kit further comprises a checklist including schedule information for each prescribed action in the pre-surgical medical prehabilitation protocol and a structure for tracking completion of each prescribed action. In some embodiments, the checklist comprises a software application loaded on a computer or a hand-held electronic device, the application executable to generate a prompt for each action to be taken by a patient in accordance with a pre-surgical medical prehabilitation protocol. In some embodiments, the application is executable to notify a user that a time period for the patient to take such action has opened. In some embodiments, the application is executable to record an input by the user indicating that the action has been completed by the patient. In some embodiments, the application is further executable to communicate user inputs to a health care provider to facilitate tracking of the patient's compliance with the pre-surgical medical prehabilitation protocol.

In some embodiments of the kit, the kit further comprises other products as specified by a health care provider, including, for example, an antibiotic ointment, a bowel prep composition, a hydration product and/or a nutritional supplement tailored to the patient's care before and after surgery.

While certain time periods are described in this disclosure as being suitable for a particular element of the POWERR programs, it is to be understood that longer time periods are also envisioned, and even preferred in some circumstances. Because each patient entering a POWERR program has a different condition or health status, longer treatments with one or more element of a POWERR program may be beneficial. For example, a patient who has good pulmonary health but poor immunonutrition would benefit from increasing the treatment time for taking immunonutrition supplements, whereas a patient who has good immunonutrition but poor pulmonary health would benefit from increasing the treatment time for pulmonary conditioning exercises or protocols and/or smoking cessation.

While the systems, methods, processes and kits disclosed herein have been described primarily for use by a patient during a period of time leading up to a surgery, it is to be understood that they also can be used in other circumstances, including, for example, for improving the health of a patient who is contending with an illness or disease or for generally improving the health of any person. Moreover, the systems, methods, processes and kits disclosed herein are beneficial even for patients who are quite fit, as such patients also can experience improved outcomes by performing a method as described herein and/or using a kit as described herein. The systems, methods, processes and kits disclosed herein advantageously engage the patient in improving his or her baseline health regardless of health status at the time of program initiation. For example, the POWERR programs described herein also have direct application in sports and human performance maintenance, injury recovery and similar challenges.

In one example of a non-surgical use that is particularly timely, the systems, methods, processes and kits described herein find advantageous use in dealing with infectious disease epidemics and pandemics, such as the COVID-19 pandemic that began in late 2019 or early 2020 and quickly spread around the world. The physical, physiologic and mental conditioning effects of the POWERR programs, including the immunonutrition and the digital engagement process described herein, have beneficial applications outside of perioperative rehabilitation. These synergistic systems, methods, processes and kits have the ability to improve recovery and resilience/tolerance to physical, metabolic, emotional and mental risks associated with stresses, such as stresses associated with the spread of infectious diseases. Such systems, methods, processes and kits have been demonstrated to yield improvements and/or stabilization and/or mitigation of such risks and also have been demonstrated to yield improvements and/or stabilization and/or mitigation of typical effects of aging, mental capacity and memory loss. Data collected to date demonstrates the synergistic effectiveness of the POWERR programs to augment physical, physiologic and immunologic protective and stress tolerance benefits. These would be very beneficial in dealing with public health events such as the COVID-19 response. For example, in the public response to COVID-19, provision of physiologic, mental and immunologic optimization and fortification as described in this disclosure would be very supportive and protective of first responders, at risk populations, and for defense against and recovery from a COVID-19 infection or infection by another infectious agent, whether viral or microbial.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention. Changes therein and other uses which are encompassed within the spirit of the invention as defined by the scope of the claims will occur to those skilled in the art.

### Example 1

A study was completed to evaluate the POWERR program. The patient engagement in this program was initiated at the time of referral to a provider. With his or her first visit, the patient underwent an assessment identifying his or her core risk factors and, specifically, malnutrition. The patient was educated by the provider in parallel with a risk assessment and optimization plan which analyzed the patient's medical history and determined steps to optimize the patient's overall health. The patient was then provided with a kit directly from the physician's office or through the pre-operative clinic. Process compliance was monitored with phone calls and other contact with the patient prior to the day of surgery and then assessed on the day of surgery. Compliance data was collected and reported through the quality process. Assessment for surgery was completed by the operating room (“OR”) staff. Through the completion of surgical recovery (whether in or out of the hospital setting), quality parameters were monitored and reported.

Our internal tracking of the impact of this program was done through direct measurement and indirect measurement interviews and feedback from providers as well as patients, and also included internal studies, standard Hospital quality indicators such as Patient Safety Indicators (PSI) including, for example, the PSI 90 composites. The program was shown to effectively support a reduction in length of stay for adult patients in the hospital system. To date, the hospital system has recovered over 300,000 days of hospitalization for surgical patients and improved its length of stay index from over 5 to nearly 1 or less. An initial internal trial engaging the immune nutrition program documented dramatic improvements in outcomes among some of the most complex patients, the patients undergoing hepatobiliary procedures. In this internal pilot study, length of stay for this high-risk patient population was reduced from 18.3 days to 6.4 days and readmissions from 19% to 9.8%. On average, the program reduces the length of stay by two days or more and has reduced surgical infectious complications by an average of 60% or more.

Review of the impact of this program by the hospital quality program was able to document a progressive reduction in total harm events. The program has also received additional validating feedback from surgeons, patients and leadership supporting the outcomes and improved recovery seen by both patients and providers. Patients who have had previous procedures without this program have reported that they would not have surgery in the future without it and that they feel substantially better and their outcomes are substantially smoother and faster in the program.

The provider research team also reported that use of the immune nutrition elements of the POWERR programs described herein resulted in substantial improvement in the recovery from surgical complications. In this regard, researchers have documented and reported that colorectal surgeons have reported no surgical site infections over a period of 18 months or more, which is a dramatic improvement over the historic rates as well as the national reported rates. Further, review specifically into the surgery-related PSI indicators, as set forth in the below table, has shown substantial reductions in these complications through the use of the POWERR programs.

| Outcome | Non-Intervention (9148) | Wellness (8010) | P value |
|---|---|---|---|
| SSI | 51 (56%) | 22 (27%) | 0.0046 |
| CLABSI | 7 (8%) | 3 (4%) | 0.3544 |
| CDI | 78 (85%) | 34 (42%) | 0.0006 |
| CAUTI | 27 (30%) | 6 (7%) | 0.0008 |
| VAE | 14 (15%) | 6 (7%) | 0.1785 |

Moreover, internal review of hospital financial records revealed a direct reduction in the average total cost of care across different DRG groups, as shown in the a sample information set forth in the below table.

| | Number of | Average | Number of | Average | Cost |
|---|---|---|---|---|---|
| | Patients | Total Cost | Patients | Total Cost | Savings |
| Enc - MS DRG_Desc | W/PAT | W/PAT | W/O PAT | W/O PAT | per patient |
| MAJOR JOINT REPLACEMENT OR | 67 | $16,956.31 | 37 | $18,354.56 | $1,398.25 |
| REATTACH OF LE W/OMCC | | | | | |
| SPINAL FUSION EXCEPT CERVICAL | 61 | $31,854.23 | 19 | $34,230.50 | $2,376.27 |
| W/O MCC | | | | | |
| MAJOR SMALL LARGE BOWEL | 58 | $22,098.25 | 56 | $24,045.51 | $1,947.27 |
| PROCEDURES W CC | | | | | |
| CARDIAC VALVE OTH MAJ CT PROC | 49 | $36,969.23 | 19 | $42,658.73 | $5,689.51 |
| W/O CARD CATH WCC | | | | | |
| MAJOR MALE PELVIC PROCEDURES | 36 | $13,302.63 | 93 | $14,941.65 | $1,639.03 |
| W/O CC/MCC | | | | | |

As will be appreciated from the descriptions herein, a wide variety of aspects and embodiments are contemplated by the present disclosure, examples of which include, without limitation, the aspects and embodiments listed below:

A method for pre-surgical medical prehabilitation of a patient prior to a date of surgery to improve post-surgical recovery that includes: (a) directing oral administration of at least one dose per day of an immunonutritional supplement per day to the patient during a first time period prior to the surgery date; (b) directing administration of at least two pulmonary preconditioning treatments per day to the patient during a second time period prior to the surgery date; (c) directing administration of a bacterial decontamination protocol to the patient during a third time period prior to the surgery date; and (d) directing oral administration of a carbohydrate loading and hydration protocol to the patient during a fourth time period prior to the surgery date.

A kit for completing a pre-surgical medical prehabilitation protocol prior to a date of surgery to improve post-surgical recovery. In some embodiments, the kit includes (a) at least five doses of an immunonutritional supplement; (b) a pulmonary preconditioning aid; (c) an anti-bacterial body wash composition; and (d) an anti-bacterial nasal cavity wash composition; (e) at least three doses of a carbohydrate loading and hydration composition.

A method in accordance with any other embodiment disclosed herein, wherein the first time period is from about three to about seven days.

A method in accordance with any other embodiment disclosed herein, wherein the second time period is from about 1 to about 30 days.

A method in accordance with any other embodiment disclosed herein, wherein the third time period is from about one to about three days.

A method in accordance with any other embodiment disclosed herein, wherein the fourth time period is from about one to about two days.

A method in accordance with any other embodiment disclosed herein, wherein the method further comprises conducting a physical assessment of the patient at least 1 day prior to the surgery date.

A method in accordance with any other embodiment disclosed herein, wherein the physical assessment comprises a nutritional evaluation.

A method in accordance with any other embodiment disclosed herein, wherein the method further comprises prescribing to the patient a pre-surgical exercise protocol during a fifth time period prior to the surgery date.

A method in accordance with any other embodiment disclosed herein, wherein the method further comprises providing to the patient at least one of educational information and assistance for smoking cessation.

A kit in accordance with any other embodiment disclosed herein, wherein the kit further comprises at least one of educational information for smoking cessation and a smoking cessation product.

A kit in accordance with any other embodiment disclosed herein, wherein the kit further comprises a nutritional supplement customized to a patient following a nutritional evaluation of the patient.

A kit in accordance with any other embodiment disclosed herein, wherein the kit comprises at least ten doses of the immunonutritional supplement.

A kit in accordance with any other embodiment disclosed herein, wherein the kit further comprises a checklist including schedule information for each prescribed action in the pre-surgical medical prehabilitation protocol and a structure for tracking completion of each prescribed action.

A kit in accordance with any other embodiment disclosed herein, wherein the checklist comprises a software application loaded on a computer or a hand-held electronic device, the application operable to generate a prompt for each action to be taken by a patient in accordance with a pre-surgical medical prehabilitation protocol.

A kit in accordance with any other embodiment disclosed herein, wherein the application is operable to notify a user that a time period for the patient to take such action has opened.

A kit in accordance with any other embodiment disclosed herein, wherein the application is operable to record an input by the user indicating that the action has been completed by the patient.

A kit in accordance with any other embodiment disclosed herein, wherein the application is further operable to communicate user inputs to a health care provider to facilitate tracking of the patient's compliance with the pre-surgical medical prehabilitation protocol.

A method or kit in accordance with any other embodiment disclosed herein, wherein the immunonutritional supplement comprises an arginine supplement.

A method in accordance with any other embodiment disclosed herein, wherein each of the pulmonary preconditioning treatments comprises a treatment selected from a deep breathing session and a spirometer treatment session.

A method in accordance with any other embodiment disclosed herein, wherein the bacterial decontamination protocol comprises a nasal antibacterial treatment from about two to about four times per day during the third time period.

A method in accordance with any other embodiment disclosed herein, wherein the bacterial decontamination protocol comprises an antibacterial body wash at least once per day during the fourth treatment period.

A method in accordance with any other embodiment disclosed herein, wherein the bacterial decontamination protocol comprises an oral antibacterial treatment.

A method in accordance with any other embodiment disclosed herein, wherein the carbohydrate loading and hydration protocol comprises directing administration of at least three doses of carbohydrate supplements per day during the fourth time period.

A method in accordance with any other embodiment disclosed herein, wherein the carbohydrate supplement comprises a source of semi-complex carbohydrates.

A method in accordance with any other embodiment disclosed herein, wherein the carbohydrate supplement comprises Maltodextrin.

A method in accordance with any other embodiment disclosed herein, wherein the carbohydrate loading and hydration protocol comprises directing administration of about 15 to about 25 ounces of G Series Gatorade® the afternoon before the surgery date, administering about 15 to about 25 ounces of G Series Gatorade® the evening before the surgery date, and administering about 7 to about 17 ounces of G Series Gatorade® the morning of the surgery date.

A method in accordance with any other embodiment disclosed herein, wherein the exercise protocol comprises walking about 20 to about 40 minutes a day for at least 5 days per week.

A method in accordance with any other embodiment disclosed herein, wherein the exercise protocol further comprises performing a cardiovascular workout about 15 to about 25 minutes at least 3 times per week.

A method in accordance with any other embodiment disclosed herein, wherein the cardiovascular workout is effective to elevate the patient's heart rate by at least 30 beats per minute for at least 15 minutes.

A method in accordance with any other embodiment disclosed herein, wherein the method further includes (a) directing oral administration of at least one dose of an immunonutritional supplement per day to the patient during a post-surgery time period; (b) directing administration of at least two pulmonary preconditioning treatments per day to the patient during the post-surgery time period; and (c) directing oral administration of a carbohydrate loading and hydration protocol to the patient during the post-surgery time period.

A kit in accordance with any other embodiment disclosed herein, wherein the set of instructions for performing pulmonary preconditioning treatments comprises instructions for performing deep breathing sessions.

A kit in accordance with any other embodiment disclosed herein, wherein the carbohydrate loading and hydration composition comprises G Series Gatorade®.

A method for optimizing the health of a person that includes (a) directing oral administration of at least one dose of an immunonutritional supplement per day to the person during a health optimizing period; (b) directing administration of at least two pulmonary preconditioning treatments per day to the person during the health optimizing period; and (c) directing oral administration of a carbohydrate loading and hydration protocol to the person during the health optimizing period.

A method in accordance with any other embodiment disclosed herein, wherein the method further includes prescribing to the person an exercise protocol during health optimizing period.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for pre-surgical medical prehabilitation of a patient prior to a surgery to improve post-surgical recovery, comprising:
directing oral administration of at least one dose of an immunonutritional supplement per day to the patient during a first time period prior to the surgery date;
directing administration of at least two pulmonary preconditioning treatments per day to the patient during a second time period prior to the surgery date;
directing administration of a bacterial decontamination protocol to the patient during a third time period prior to the surgery date; and
directing oral administration of a carbohydrate loading and hydration protocol to the patient during a fourth time period prior to the surgery date.

2. The method of claim 1 wherein the first time period is from about three to about seven days, wherein the second time period is from about 1 to about 30 days, wherein the third time period is from about one to about three days, and wherein the fourth time period is from about one to about two days.

3. The method of claim 1 wherein the immunonutritional supplement comprises an arginine supplement.

4. The method of claim 1 wherein each of the pulmonary preconditioning treatments comprise a treatment selected from a deep breathing session and a spirometer treatment session.

5. The method of claim 1 wherein the bacterial decontamination protocol comprises a treatment selected from the group consisting of a nasal antibacterial treatment from about two to about four times per day during the third time period, an antibacterial body wash at least once per day during the fourth treatment period, an oral antibacterial treatment and combinations thereof

6. The method of claim 1 wherein the carbohydrate loading and hydration protocol comprises an action selected from the group consisting of directing administration of at least three doses of carbohydrate supplements per day during the fourth time period; directing administration of about 15 to about 25 ounces of G Series Gatorade® the afternoon before the surgery date, about 15 to about 25 ounces of G Series Gatorade® the evening before the surgery date, and about 7 to about 17 ounces of G Series Gatorade® the morning of the surgery date; and combinations thereof.

7. The method of claim 6 wherein the carbohydrate supplement comprises a supplement selected from the group consisting of a source of semi-complex carbohydrates and Maltodextrin.

8. The method of claim 1, further comprising an action selected from the group consisting of prescribing to the patient a pre-surgical exercise protocol during a fifth time period prior to the surgery date, providing to the patient at least one of educational information and assistance for smoking cessation, conducting a physical assessment of the patient at least 1 day prior to the surgery date, and combinations thereof.

9. The method of claim 1, further comprising:
directing oral administration of at least one dose of an immunonutritional supplement per day to the patient during a post-surgery time period;
directing administration of at least two pulmonary preconditioning treatments per day to the patient during the post-surgery time period; and
directing oral administration of a carbohydrate loading and hydration protocol to the patient during the post-surgery time period.

10. A kit for completing a pre-surgical medical prehabilitation protocol prior to a date of surgery to improve post-surgical recovery, comprising:
at least five doses of an immunonutritional supplement;
a member selected from the group consisting of a spirometer, a set of instructions for performing pulmonary preconditioning treatments and a combination thereof;
an anti-bacterial body wash composition;
an anti-bacterial nasal cavity wash composition; and
at least three doses of a carbohydrate loading and hydration composition.

11. The kit of claim 10, further comprising a member selected from the group consisting of educational information for smoking cessation, a smoking cessation product, a nutritional supplement customized to a patient following a nutritional evaluation of the patient and combinations thereof.

12. The kit of claim 10, further comprising a checklist including schedule information for each prescribed action in the pre-surgical medical prehabilitation protocol and a structure for tracking completion of each prescribed action.

13. The kit of claim 12 wherein the checklist comprises a software application loaded on a computer or a hand-held electronic device, the application operable to generate a prompt for each action to be taken by a patient in accordance with a pre-surgical medical prehabilitation protocol.

14. The kit of claim 13 wherein the application is operable to notify a user that a time period for the patient to take such action has opened.

15. The kit of claim 13 wherein the application is operable to record an input by the user indicating that the action has been completed by the patient.

16. The kit of claim 13 wherein the application is further operable to communicate user inputs to a health care provider to facilitate tracking of the patient's compliance with the pre-surgical medical prehabilitation protocol.

17. A method for optimizing the health of a person, comprising:
directing oral administration of at least one dose of an immunonutritional supplement per day to the person during a health optimizing period;
directing administration of at least two pulmonary preconditioning treatments per day to the person during the health optimizing period;
directing oral administration of a carbohydrate loading and hydration protocol to the person during the health optimizing period.

18. The method of claim 17 wherein the health optimizing period is from about 1 to about 30 days.

19. The method of claim 17, further comprising prescribing to the person an exercise protocol during health optimizing period.

20. The method of claim 17, further comprising providing to the person at least one of educational information and assistance for smoking cessation.
